(19) United States
(12) Patent Application Publication
(10) Pub. No.: US 2022/0162537 A1
(43) Pub. Date: May 26, 2022

(54) FLUID SYSTEM FOR PRODUCING EXTRACELLULAR VESICLES COMPRISING A THERAPEUTIC OR IMAGING AGENT AND METHOD THEREOF

(71) Applicants: CENTRE NATIONAL DE LA RECHERCHE SCIENTIFIQUE, Paris (FR); UNIVERSITE DE LORRAINE, NANCY (FR); UNIVERSITÉ DE PARIS, Paris (FR)

(72) Inventors: Iris MARANGON, PARIS (FR); Marie MILLARD, VANDOEUVRE-LES-NANCY (FR); Lina BOLOTINE, VANDOEUVRE-LES-NANCY (FR); Amanda SILVA, IGNY (FR); Alice GRANGIER, GENTILLY (FR); Florence GAZEAU, LE KREMLIN BICETRE (FR); Claire WILHELM, VELIZY-VILLACOUBLAY (FR); Max PIFFOUX, LYON (FR)

(73) Assignees: CENTRE NATIONAL DE LA RECHERCHE SCIENTIFIQUE, Paris (FR); UNIVERSITÉ DE LORRAINE, NANCY (FR); UNIVERSITE DE PARIS, Paris (FR)

(21) Appl. No.: 17/418,971

(22) PCT Filed: Dec. 27, 2019
(86) PCT No.: PCT/FR2019/053308

§ 371 Date: Jan. 27, 2022

(30) Foreign Application Priority Data
Dec. 28, 2018 (FR) FR1874313

(51) Int. Cl. C12M 1/06; C12N 15/86; C12M 3/06; A61K 45/06; C12M 1/34; C12M 1/00

(52) U.S. Cl. CPC C12M 27/02; C12N 15/86; C12M 47/10; A61K 45/06; C12M 41/42; C12M 23/16

(57) ABSTRACT

A fluidic system for loading a therapeutic or imaging agent into the lumen of extracellular vesicles from producer cells, including at least one container, a liquid medium contained in the container, producer cells, a liquid medium stirrer and a device for controlling the speed of the stirrer suitable for the growth of the producer cells, wherein it also includes a device for controlling the speed of the stirrer and the stirrer, of which the shape and dimensions of the container are suitable for generating a turbulent flow of the liquid medium in the container for exerting shear stresses on the producer cells in order to carry out the loading of a therapeutic or imaging agent into the lumen of the extracellular vesicles produced simultaneously by the fluidic system.

### FIELD

The invention generally relates to the production of extracellular vesicles and the loading of at least one therapeutic or imaging agent. More specifically, the invention relates to a system for loading extracellular vesicles from producer cells and a therapeutic or imaging agent, to a method for loading a therapeutic or imaging agent and for recovering such vesicles and vesicles produced by such a system, the extracellular vesicles may for example be of interest as vectors for delivery of therapeutic or imaging agents, as an alternative to cell therapy and in regenerative medicine.

### BACKGROUND

The cells are known to release extracellular vesicles in their environment, for example, in vivo, in the biological fluids of an organism. The extracellular vesicles have been identified as effective means for delivering drugs, in a personalized or targeted manner, into the human body. They first have a native biocompatibility and an immune tolerance. They can also internalize theranostic nanoparticles, making it possible both to image certain parts of the body and to deliver active ingredients having therapeutic functions. The extracellular vesicles also have a cell-to-cell communication function: they allow, for example, to transport lipids, membrane and cytoplasmic proteins and/or nucleotides of the cell cytoplasm, such as mRNA, microRNAs or long non-coding RNAs, between different cells.

In particular, the use of extracellular vesicles can solve problems known in the therapeutic use of cells, such as cell replication, differentiation, vascular occlusions, risk of rejection and difficulties in storage and freezing. There is therefore an industrial need for the production of functionalized cellular vesicles (ie, loaded with a compound of interest) in amounts sufficient for therapeutic use, in particular in replacement or in addition to cell therapies. The unique properties of extracellular vesicles (EVs) and their biological tolerance are now considered to be advantages for delivering biologically active macromolecules, while protecting enzymes circulating in body fluids.

The two main challenges for therapeutic use are (i) the generation of EVs in an amount sufficient for clinical use and (ii) the efficiency of loading biologically active compounds of interest.

Today, two major types of loading techniques are described, (i) the biological modifications of the parent cells, or (ii) the loading of EVs after their production by physical means. With regard to the loading of the parent cells, it has been described techniques for spontaneous loading or by transfection. For example, cells have been designed to express an RVG peptide on the extracellular portion of the lamp2b protein overexpressed on EVs (Alvrez-Erviti et al, 2011). However, such a strategy for transfection of the mother cells by plasmids encoding the lamp2b fused to the peptide of interest takes time, poses challenges, and can be difficult to comply with a scalable process of good handling practices (Good Manufacturing Practice or GMP). In parallel, the electroporation method is the method which can be used for the loading of EVs after production. This method was used to charge various compounds in EVs such as siRNA (Shtam et al, 2013) DNA (Lamihhane et al, 2015) and doxorubicin (Tian et al, 2014). However, the siRNA charge has been found to be inefficient due to the formation of siRNA aggregates. International application WO 2004/083379 also describes a method for loading an exogenous agent into extracellular vesicles comprising the application of an electrical charge. Another method for obtaining functionalized EVs (ie, vesicles loaded with a compound of interest) consists in destroying EVs in order to functionalize them (Haney et al, 2015). This method is easy to implement, but does not make it possible to preserve the bladder structure, which causes the loss of the asymmetry of the membrane and of the proteins with poor rearrangement of the membrane proteins. Smyth et al disclose a method for loading EVs by click chemistry. This method consists in loading the membrane proteins of EVs with a specific functional group in order to bind to these proteins the compound of interest. However, this method does not make it possible to charge the lumen of the vesicles.

It is therefore necessary to provide a method for obtaining functionalized extracellular vesicles (EVs) in which the topology and the original properties of the vesicles are preserved and making it possible to charge the lumen of the vesicles. It is also necessary for this method to charge EVs with all kinds of compounds, with any type of sample volume. Finally, it is also necessary for this method to load EVs for clinical use compatible with the Good Manufacturing Practice (GMP), or easy to implement with a reduced number of steps, and allows a large quantity of charged EVs containing a large amount of cargo, etc.

### SUMMARY

It is an object of the invention to provide a solution for loading therapeutic and/or imaging agents into the membrane or in the lumen of the extracellular vesicles and thus functionalising extracellular vesicles in large quantity from producer cells, more rapidly and more efficiently than with the known methods, under conditions complying with the GMP standards. Another object of the invention is to propose a solution for increasing the yield of the loading system of therapeutic and/or imaging agent in vesicles, i.e. the ratio of the number of vesicles loaded with therapeutic and/or imaging agent and the number of non-charged vesicles. Another object of the invention is to propose a solution for loading, producing and recovering extracellular vesicles loaded with therapeutic and/or imaging agent in continuous way or in batch. Finally, another object of the invention is to simplify the structure of the fluidic system for loading and producing vesicles loaded with therapeutic and/or imaging agent and to reduce its manufacturing cost.

Thus, the invention proposes a solution for loading the extracellular vesicles produced by the fluidic system of a therapeutic agent and/or imaging agent.

In particular, an object of the invention is a fluidic system for loading a therapeutic and/or imaging agent into the membrane or in the lumen of the extracellular vesicles (EVs) from producer cells, comprising at least one container, a liquid medium contained by the container, producer cells, a liquid medium stirrer suitable for the growth of the producer cells characterized in that it also comprises means for controlling the speed of the stirrer, the stirrer, and the dimensions of the container being suitable for generating a turbulent flow of the liquid medium in the container in order to exert shear stresses on the producer cells in order to carry out the loading of a therapeutic and/or imaging agent into the membrane or in the lumen of the extracellular vesicles (EV) produced simultaneously by the fluidic system.

It is understood that with such a system, it is possible to produce vesicles loaded with therapeutic and/or imaging agent in large quantity, and in a system suitable for GMP standards. It also comprises that such a system is simpler and less expensive to manufacture than known systems for loading and functionalizing extracellular vesicles, the length of Kolmogorov of the flow being less than 100 μm.

The invention is advantageously completed by the following features, taken individually or in any of their technically possible combinations:
the length of Kolmogorov of the flow being less than or equal to 100 μm, and preferentially less than or equal to 70 μm; more preferably less than or equal to 60 μm;
the fluidic system comprises an outlet and a connector connected to the outlet, the connector being capable of comprising liquid medium and extracellular vesicles;
the fluidic system comprises microcarriers to which adherent producer cells will be attached;
the stirrer is preferably a rotary stirrer, the rotation speed or speeds of which, the shape, the size are adapted, with the shape and dimensions of the container, to the generation of a turbulent flow of the liquid medium in the container;
the microcarriers are microbeads, the diameter of the microbeads being between 100 μm and 300 μm;
the fluidic system comprises a separator of extracellular vesicles, fluidly connected to the container so as to be capable of reintroducing into the container a liquid medium depleted in extracellular vesicles (EV). The fluidic system may comprise a closure means upstream of the separator for closing or opening the connector and thus obtaining a system for recovering vesicles continuously or discontinuously.

Another object of the invention is a method for loading a therapeutic and/or imaging agent into the membrane or in the lumen of the extracellular vesicles (EV) from producer cells, comprising:
means for controlling the speed of an stirrer driving a turbulent flow of a liquid medium in a container to exert shear stresses on the producer cells in order to carry out the loading of a therapeutic and/or imaging agent into the membrane or in the lumen of the extracellular vesicles (EV), the length of Kolmogorov of the flow being less than 100 μm, preferably less than or equal to 70 μm, more preferably less than or equal to 60 μm in a container, the container comprising an outlet, the liquid medium comprising the therapeutic and/or imaging agent, producing cells, and
a collection of the liquid medium comprising extracellular vesicles (EV) at the outlet of the container.

The process is advantageously completed by the following features, taken individually or in any of their technically possible combinations:
the liquid medium is stirred for more than twenty minutes;
the stirrer is controlled to drive a flow of the liquid medium constant, intermittent, of increasing or decreasing intensity, the length of Kolmogorov of the flow being less than 100 μm, preferentially less than or equal to 70 μm, more preferentially less than or equal to 60 μm;
a separator which makes it possible to deplete a portion of the liquid medium collected at the outlet of the container into extracellular vesicles, and the liquid thus depleted being reintroduced into the portion of the liquid medium in the container.
a step of ultracentrifugation or tangential filtration after collection to separate the vesicles, the producer cells and the therapeutic and/or imaging agents in the liquid medium.
the extracellular vesicles (EV) at the outlet of the container comprise a mixture of extracellular vesicles loaded with a therapeutic and/or imaging agent or non-charged extracellular vesicles.
the flow allows, at the same time, to charge the therapeutic agent and produce the extracellular vesicles (EV) in a container.

The loading of the extracellular vesicles according to the method of the invention can also be carried out independently of their production.

Thus, an object of the invention is a method for loading at least one therapeutic and/or imaging agent by directly using a suspension of previously produced extracellular vesicles. An object of the invention is therefore a method for loading into the membrane or in the lumen of extracellular vesicles (EV), comprising:
providing extracellular vesicles in the liquid medium (5),
a control of the speed of a stirrer causing a turbulent flow of a liquid medium in a container to exert shear stresses on the vesicles in order to carry out the loading of a therapeutic and/or imaging agent into the membrane or the lumen of the extracellular vesicles, the length of Kolmogorov of the flow being less than 100 μm, the liquid medium comprising the therapeutic and/or imaging agent.

Another object of the invention is a method for loading at least one therapeutic and/or imaging agent into the membrane or in the cytoplasm of producer cells, comprising:
a control of the speed of an stirrer causing a turbulent flow of a liquid medium in a container to exert shear stresses on the producer cells in order to carry out the loading of a therapeutic and/or imaging agent into the membrane or in the cytoplasm of the producer cells, the length of Kolmogorov of the flow being less than or equal to 100 μm, preferably less than or equal to 70 μm, more preferably less than or equal to 60 μm in a container, the container comprising an outlet, the liquid medium comprising the therapeutic and/or imaging agent, producing cells, and
a collection of the liquid medium comprising extracellular vesicles at the outlet of the container.

This method for loading producing cells is advantageously completed by the following features, taken individually or in any of their technically possible combinations:
the liquid medium is stirred for more than twenty minutes;
the stirrer is controlled to drive a flow of the liquid medium constant, intermittent, of increasing or decreasing intensity, the length of Kolmogorov of the flow being less than 100 μm, preferentially less than or equal to 70 μm, more preferentially less than or equal to 60 μm.

As demonstrated by experiments, the system or methods of the invention make it possible to obtain charged vesicles and/or producer cells of at least one therapeutic and/or imaging agent at particularly higher concentrations (the measured increases vary from 39% to 592%) compared to the passively charged/produced vesicles and/or cells. Thus, said producer cells and extracellular vesicles are of particular interest and therefore constitute an object of the present invention. The vesicles of the invention are more particularly of interest as a vector of at least one therapeutic and/or imaging agent. These uses also constitute an object of the invention.

More particularly, the vesicles loaded according to the methods of the invention have improved pharmacodynamic and therapeutic properties compared to liposomal formulations, as shown by the data relating to temoporfin. Thus, a particular object of the invention is the method according to the invention in any one of its embodiments, an extracellular vesicle, producer cell obtained according to this method for which said therapeutic agent is selected from temoporfin, amphotericin B, daunorubicin, irinotecan, vincristine, cytarabine.

The term “extracellular vesicle” generally designates a vesicle endogenously released by a producer cell, the diameter of which is between nm and 5000 nm. An extracellular vesicle, in particular, corresponds to an exosome and/or a microvesicle and/or a cellular apoptotic body. It is known from the art that the extracellular vesicles contain the membrane and/or cytoplasmic markers from the producer cells. These markers make it possible to identify and characterize these vesicles and are responsible for their functionality. As shown in the experimental part, the vesicles according to the invention are more effective than the liposomes, for example, and enable an improvement in pharmacokinetics/pharmacodynamics (PK/PD) of the molecules they vectorize.

The term “producer cell” generally denotes either a cell which is not adherent to a medium, or an adherent cell on a medium and which can divide and multiply. According to another aspect of the invention, the term “producer cells” designates cells of human, animal or vegetable origin or originating from bacteria or other microorganisms capable of secreting extracellular vesicles. In the case of adherent cells, these can be adherent to microcarriers themselves suspended in the liquid culture medium. According to another aspect of the invention, the term “producer cells” designates cell aggregates. The term “cell aggregates” designates an assembly of a plurality of producer cells that are firmly adhered to each other. A mild mixture created by the stirrer allows the adherent producer cells to remain suspended in the liquid culture medium.

The terms “microcarrier” and “microsupport” denote a spherical matrix allowing the growth of producer cells adherent to its surface or inside and whose size is between 50 μm and 500 μm, and preferably between 100 μm and 300 μm. The microcarriers are generally beads, the density of which is chosen substantially close to that of the liquid culture medium of the producer cells. Thus, a mild mixture allows the beads to remain suspended in the liquid culture medium.

The term “therapeutic agent” or “imaging agent” generally designates any agent of interest which can be loaded, inserted into the extracellular vesicles. These agents may be therapeutic, imaging, nanoparticles for therapeutic purposes, imaging purposes, etc. As show the experimental data, the invention is able to allow for improved loading of a wide variety of therapeutic or imaging agent size, such as small molecules, polymers, proteins, etc. regardless of the type of producer cells. Thus, due to the possible diversity of therapeutic agent incorporated by the method according to the invention and thus vectorable by the extracellular vesicles of the invention and also due to the wide variety of usable producer cells, the vesicles according to the invention are usable for any kind of therapy; for example, and in a non-limiting manner, it can be the therapy of infectious, inflammatory, immunological, metabolic, cancerous, genetic, degenerative diseases or secondary to surgeries or traumas. The vectorization of molecules of low bioavailability is particularly preferred. Likewise, a wide variety of imaging agents and/or tracers can be loaded into the extracellular vesicles according to the invention, for example, and in a non-limiting manner, fluorescent agents, luminescent agents, radioactive isotopes, contrast agents with magnetic, plasmonic, acoustic or radio-opaque properties. It may also be proteins or other biological or synthetic molecules coupled to these agents including targeting agents in order to change the biodistribution of the vesicles.

The term “stirrer” generally designates a means for agitating the liquid. This can be a mechanical part at least partially in contact with a part of the liquid and which makes it possible to put this liquid into motion. This is for example the case with a rotary stirrer. A person skilled in the art knows that it is possible to use numerous variants to produce a liquid movement and a mixture, either by varying the shape characteristics of the rotary stirrer, or by using other types of actions, either alone or in conjunction, in the manner of inducing movement in the liquid. Thus, a “shake-flash” reactor uses a shaking motion to induce movement of the liquid and its mixture; an “air-lift” reactor uses the injection of gas bubbles into the liquid to produce a movement of the liquid and its mixture. Other reactor configurations exist which optionally take advantage of the use of a flexible enclosure to contain the liquid, associated with a deformation of the flexible enclosure to produce a liquid movement and a mixture. Likewise, a mixing movement can be obtained by means of a cyclic variation in inclination of the reactor with respect to gravity, so as to create waves in the liquid, and promote flow and mixing. Finally, static structures present in the reactor, for example baffles, or structures forming partial barriers to the movement of the liquid, as used in a static mixer, can naturally also be used.

The term “stirrer” should be understood in an extremely general direction, which is that of any means or a combination of any means for generating the combination of a flow, the mixture of the medium, and the generation of turbulence in a liquid medium.

### BRIEF DESCRIPTION OF THE DRAWINGS

Other features and advantages will become apparent from the following description, which is purely illustrative and non-limiting, and must be read nest to the attached figures, among which:

FIG. 1 schematically illustrates a fluidic system for loading a therapeutic and/or imaging agent into the membrane or in the lumen of extracellular vesicles from suspension-producing cells;

FIG. 2 schematically illustrates a fluidic system for loading a therapeutic and/or imaging agent into the membrane or in the lumen of extracellular vesicles from adherent producer cells and comprising microcarriers;

FIGS. 3A, 3B respectively illustrate the size distribution of EVs obtained by NTA, the morphological analysis by cryo-TEM;

FIG. 4 illustrates the fluorometry analysis of the vesicles loaded with mTHPC.

FIG. 5 illustrates the biodistribution of a commercially available liposomal formulation of mTHPC (Foslip®, A and B) and vesicles with mTHPC (C and D) studied according to the intensity of fluorescence in selected tissues as a function of time after intravenous injection (0.3 mg/kg of the agent of interest) in mice carrying HT29 tumors.

FIG. 6 illustrates the biodistribution of the charged vesicles according to the invention with mTHPC (A and B) studied according to the intensity of the fluorescence in selected tissues as a function of time after intravenous injection (0.3 mg/kg of the agent of interest) in mice bearing HT29 tumors.

FIG. 7 illustrates the plasma concentration of mTHPC expressed as a function of time after intravenous injection of the liposomal formulation of mTHPC or mTHPC-EV (0.3 mg/kg mTHPC) in mice carrying HT29 tumors.

FIG. 8 illustrates Kaplan-Meier diagrams of HT29 tumor growth retardation after treatment with free mTHPC; the liposomal formulation of mTHPC (mTHPC Liposome) and mTHPC vesicles with laser activation of the drug (photodynamic therapy), compared to the same groups without laser activation (control, dotted lines).

FIG. 9 illustrates the impact of the length of Kolmogorov on the doxorubicin loading of HUVEC (cell concentration in doxorubicin).

FIG. 10 illustrates the impact of Kolmogorov length on the doxorubicin loading of HUVEC extracellular vesicles (doxorubicin concentration for 10⁶ vesicles).

FIG. 11 illustrates the impact of the length of Kolmogorov on the number of extracellular vesicles of HUVEC formed, measured in NTA (grey bars) or an arbitrary unit of luminescence of the luciferase. A comparable number of vesicles is obtained for a Lₖ of 48 μm, whether in the absence or in the presence of the cargo (FITC-dextran 70 kDa).

FIG. 12 illustrates the impact of the length of Kolmogorov on the FITC-dextran loading 70 kDa of HUVEC extracellular vesicles measured by FITC fluorescence (arbitrary units) contained in the vesicles.

FIG. 13 illustrates the impact of the length of Kolmogorov on the loading into FITC-dextran 70 kDa (FIG. 13A) or FITC-dextran 10 kDa (FIG. 13B) as a function of the size of the extracellular vesicles of HUVEC formed, measured by the fluorescence of the FITC (arbitrary units) contained in the vesicles. Black bars, loading at a Lk=245 μm, white bars, loading at a Lk=48 μm.

### DETAILED DESCRIPTION

### Theoretical Elements

The length of Kolmogorov (or dimension of Kolmogorov or length of eddy) is the length from which the viscosity of a fluid makes it possible to dissipate the kinetic energy of a flow of this fluid. In practice, the length of Kolmogorov corresponds to the size of the smallest vortices in a turbulent flow. This length Lₖ is calculated in the publication of Kolmogorov (Kolmogorov, A. N., 1941, January, The local structure of turbulence in incompressible viscous fluid for very large Reynolds numbers, In Dokl. Akad. Nauk, SSSR, Vol. 30, No. 4, pp. 301-305) and described by the following formula (I):

Lₖ=v^{3/4}·ε^{-1/4} (I)

in which v is the kinematic viscosity of the flowing liquid medium and £ is the average rate of energy dissipation in the fluid per unit mass (or energy injection rate in the fluid).

Zhou et al. (Zhou, G., Kresta, S. M., 1996, Impact of tank geometry on the maximum turbulence energy dissipation rate for impellers, AIChE journal, 42(9), 2476-2490) describe the relationship between average £ and the geometry of a container in which a liquid medium is stirred by paddlewheel type stirrer. This relationship is given by the following formula (II):

$$ɛ = \frac{{N}_{p} \cdot {D}^{5} \cdot {N}^{3}}{V} \qquad ( II )$$

in which Nₚ is the number of power (or number of Newton) that is the size of the stirrer in the liquid medium, D is the diameter of the stirrer (in meter), N is the speed of rotation (in number of revolutions per second) and V is the volume of liquid medium (in cubic meter). This relationship is used for the calculation of the average ε corresponding to the geometry of a container and a stirrer used for the implementation of the invention. The number of power Nₚ is given in a known manner by formula (III):

$${N}_{p} = \frac{P}{{N}^{3} {D}^{5} ρ} \qquad ( III )$$

in which P is the power supplied by the stirrer, and p is the density of the liquid medium. Formula (III) can be adjusted as described in Nienow et al. (Nienow, A. W., Et Miles, D., 1971, Impeller power numbers in closed vessels, Industrial Et Engineering Chemistry Process Design and Development, 10(1), 41-43) or Zhou et al. (Zhou, G., Kresta, S. M., 1996, Impact of tank geometry on the maximum turbulence energy dissipation rate for impellers, AIChE journal, 42(9), 2476-2490) as a function of the Reynolds number of the flow of the liquid medium. It is also possible to calculate the Reynolds number of the system by the following formula (IV):

$$R e = \frac{N \cdot {D}^{2}}{v} \qquad ( IV )$$

Alternatively, a person skilled in the art of his general knowledge and with alternative calculation modes can calculate the length of Kolmogorov based on an average energy dissipation rate per unit volume. In any state, the calculation presented above is only one way among others known to a person skilled in the art to calculate the length of Kolmogorov and illustrates one embodiment of the invention without limiting the scope of the invention. Generally, for a selected container and stirrer, a person skilled in the art will know to apply the Nₚ provided by the provider of the stirrer and thus determine how to obtain a desired Lₖ.

### General Architecture of the Fluidic System

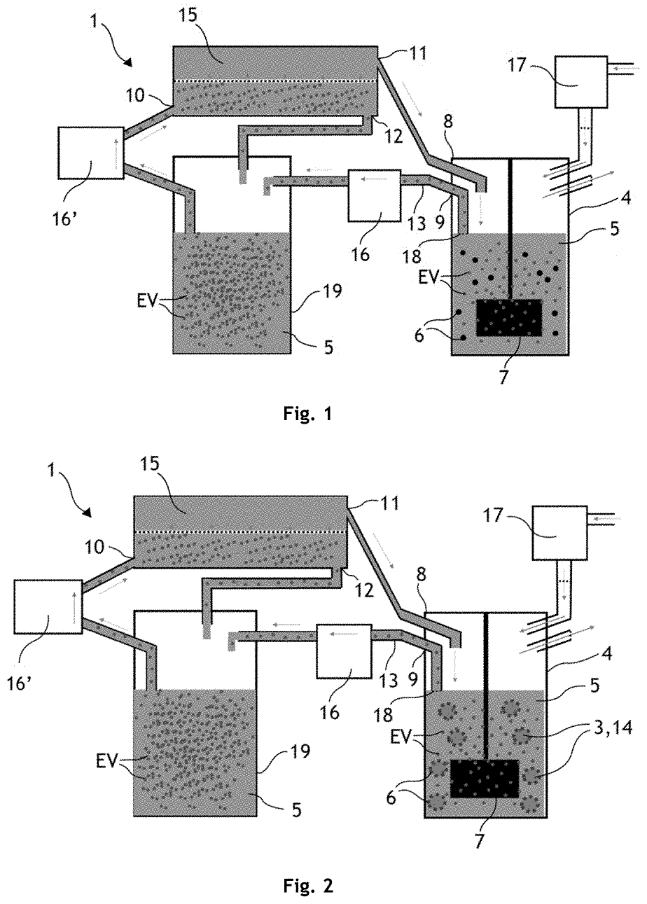

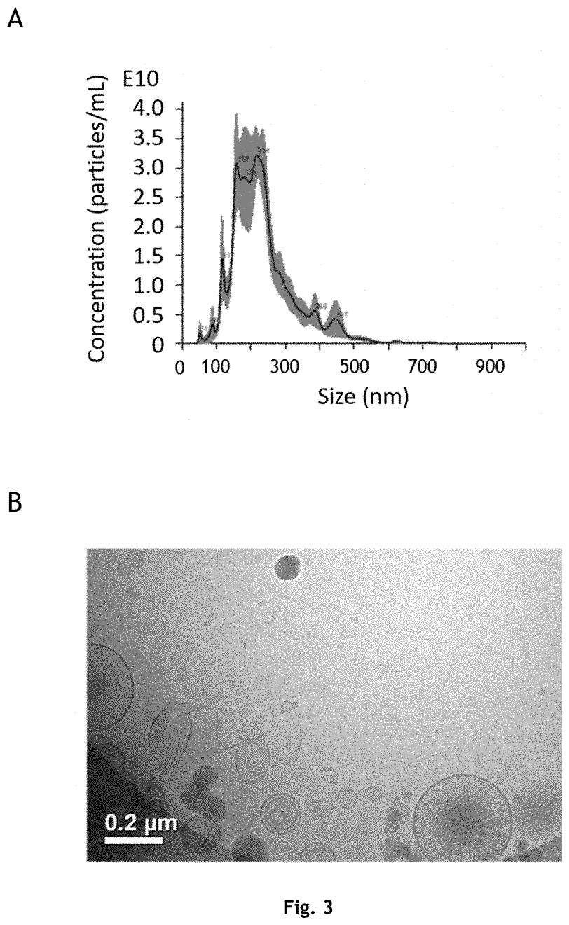

FIGS. 1 and 2 schematically illustrate a fluidic system (1) for the loading of extracellular vesicles (EV). The fluid system (1) for loading extracellular vesicles (EV) aims to produce a large quantity of extracellular vesicles (EV) loaded in a container (4). However, the invention is not limited to this embodiment and may comprise a series of containers (4) fluidly connected in parallel or in series.

The container (4) contains a liquid medium (5). The container (4) may in particular be a tank, a flange, for example made of glass or plastic, or any other container suitable for containing a liquid medium (5). The volume of the container (4) is one of the factors making it possible to produce extracellular vesicles (EV) in large quantity: this volume may be between 50 mL and 500 L, preferably between 100 mL and 100 L, and preferably between 300 mL and 40 L. The volume of the container (4) illustrated schematically in FIG. 1 or 2 is 1 L in the non-limiting example of the embodiment shown in FIG. 1, which allows the continuous separation of the vesicles produced, the liquid medium (5) can be extracted from the container (4) by a first pump (16), via a connector (13), so as to transport the liquid medium (5) into a collector (19). Another pump (16′) makes it possible to supply the liquid medium (5) contained in the collector (19) to the inlet (10) of the separator (15), via another connector. The first outlet (11) of the separator (15) is connected to the container (4) via a connector, so as to reintroduce liquid medium 5 depleted in extracellular vesicles (EV) into the container (4). The second outlet (12) of the separator (15) is connected to the collector (19) via a connector, so as to enrich the liquid medium (5) contained in the collector (19) into extracellular vesicles (EV). Alternatively, the inlet (10) of the separator (15) can be directly connected to the outlet (9) of the container (4) (or via a first pump (16)). The first outlet (11) of the separator (15) is connected to the container (4) and the second outlet (12) of the separator (15) is connected to the collector (19). Several separators may also be arranged in series to vary the degree of separation of extracellular vesicles EV in the liquid medium 5, and/or in parallel to adapt the flow rate of liquid medium 5 in each separator 15 to the flow rate of a first pump 16. A filter (18) can be arranged at the outlet (9) so as to filter the producer cells (6) and the cell debris when extracting extracellular vesicles EV from the container (4).

The container (4) typically comprises one or more gas inlets and one or more gaseous outlets, through which an atmosphere comprising concentrations of air, N₂, O₂ and CO₂ suitable for cell culture can flow, for example comprising 5% CO₂. This atmosphere may be from a suitable gas injector/mixer or a CO₂ controlled atmosphere oven. A pump (17) is used to control this gas flow in the container (4). The container (4) also comprises an outlet (9) capable of comprising liquid medium (5) and extracellular vesicles (EV). This outlet can be supplemented with a means for separating and/or filtering the cells in suspension making it possible not to recover cells suspended outside the container (4). This outlet (9) makes it possible to extract the produced extracellular vesicles (EV) out of the container (4). The container (4) may also comprise at least one inlet (8) adapted to introduce the liquid medium (5) into the container (4).

The liquid medium (5) may be generally a saline solution, for example isotonic. Preferably, the liquid medium 5 is either a culture liquid medium with the addition of compounds allowing the culture of the cells of interest, or a medium supplemented with serum or platelet lysate previously purified from the extracellular vesicles or a serum-free medium, making it possible not to contaminate the extracellular vesicles (EV) produced by the fluidic system 1 by proteins or other vesicles originating from a serum. A serum-free DMEM liquid medium (5) can be used. The maximum volume of liquid medium (5) is determined in part by the container (4). This maximum volume may also be between 50 mL and 500 L, preferably between 100 mL and 100 L, and more preferably between 300 mL and 40 L. The minimum volume of liquid medium (5) contained by the container (4) is partly determined by the choice of the stirrer (7) making it possible to agitate the liquid medium (5).

The fluidic system (1) may comprise, according to a particular embodiment, the microcarriers (3) suspended in the liquid medium (5). The microcarriers are particularly advantageous when the producer cells (6) are adherent cells. The microcarriers (3) may be microbeads (14), for example Dextran, each microbead (14) being able to be covered with a layer of collagen or other material necessary for the culture of cells. Other materials may be used for the manufacture of the microcarriers (3), such as glass, polystyrene, polyacrylamide, collagen and/or alginate. Generally, all of the microcarriers (3) suitable for cell culture is suitable for the production of extracellular vesicles (EV). The density of the microcarriers (3) can be, for example, slightly greater than that of the liquid medium (5). The density of the microbeads (14) in Dextran is for example 1,04. This density allows the microbeads (14) to be suspended in the liquid medium (5) by slightly stirring the liquid medium (5), the drag of each microcarrier (3) in the liquid medium (5) being dependent on the density of the microcarrier (3). The maximum size of the microcarriers (3) may be between 50 μm and 500 μm, preferably between 100 μm and 300 μm, and preferentially between 130 μm and 210 μm.

The microcarriers (3) may, for example, be microbeads (14) of the Cytodex 1 type (registered trademark). A powder formed by these microbeads (14) may be rehydrated and sterilized prior to use. The rehydration can be used in PBS and then transferred to a culture medium (for example DMEM) without serum, in which the microbeads are kept at 4° C. before use.

The fluidic system (1) also comprises producer cells (6). The producer cells (6) can be according to one embodiment, adherent cells on the microcarriers (3) or in another embodiment, the cells in suspension. The extracellular vesicles EV are loaded and produced by the fluidic system (1) from these producer cells (6) (adherent or suspended).

The producer cells (6) can be cultured, prior to the loading and production of extracellular vesicles (EV) loaded by the fluidic system (1), on the surface of the microcarriers (3) in a cell culture medium suitable or suspended in a cell culture medium suitable for suspended cells. Thus, no cell transfer is required between the culture of the producer cells (6) and the loading of the extracellular vesicles (EV), thereby avoiding contamination and simplifying the process as a whole. According to one embodiment, the majority of the producer cells (6) are adherent to the surface of the microcarriers (3), even if a minor proportion of producer cells (6) can be peeled off, for example by stirring the liquid medium (5). The other producer cells are then suspended in the liquid medium (5) or sedimented at the bottom of the container (4). According to a particular embodiment of the invention, at least 50% of the producer cells (6) are adherent to the surface of the microcarriers (3), preferably at least 60% of the producer cells (6) are adherent to the surface of the microcarriers (3), preferably at least 70% of the producer cells (6) are adherent to the surface of the microcarriers (3), preferably at least 80% of the producer cells (6) are adherent to the surface of the microcarriers (3), preferably at least 85% of the producer cells (6) are adherent to the surface of the microcarriers (3), preferably at least 90% of the producer cells (6) are adherent to the surface of the microcarriers (3), preferably at least 95% of the producer cells (6) are adherent to the surface of the microcarriers (3), preferably at least 96% of the producer cells (6) are adherent to the surface of the microcarriers (3), preferably at least 97% of the producer cells (6) are adherent to the surface of the microcarriers (3), preferably at least 98% of the producer cells (6) are adherent to the surface of the microcarriers (3), preferably at least 99% of the producer cells (6) are adherent to the surface of the microcarriers (3), preferably 100% of the producer cells (6) are adherent to the surface of the microcarriers (3). Thus, less than 50% of the producer cells (6) are suspended, preferably less than 40% of the producer cells (6) are suspended, preferably less than 30% of the producer cells (6) are suspended, preferably less than 20% of the producer cells (6) are suspended, preferably less than 15% of the producer cells (6) are suspended, preferably less than 10% of the producer cells (6) are suspended, preferably less than 5% of the producer cells (6) are suspended, preferably less than 4% of the producer cells (6) are suspended, preferably less than 3% of the producer cells (6) are suspended, preferably less than 2% of the producer cells (6) are suspended, preferably less than 1% of the producer cells (6) are suspended, preferably the producer cells (6) are not suspended. Preferably, the fluidic system (1) is adapted so as to generate a gentle agitation making it possible to homogenize the producer cells 6 in the medium liquid (5) within the container (4). Generally, any type of producer cells (6) may be used, including non-adherent producer cells. The suspension-producing cells are then suspended in the liquid medium (5) or sedimented at the bottom of the container (4).

The container (4) also comprises a stirrer (7) for agitating the liquid medium (5). The stirrer (7) may be a blade such as an impeller, the blades of which are at least partially immersed in the liquid medium (5), and moved by a transmission of magnetic or mechanical forces. The stirrer (7) may also be a liquid medium infusion system (5) at a flow rate sufficient to agitate the liquid medium (5) contained by the container, or a rotary wall system (e.g., arranged on rollers). The stirrer (7) may alternatively be of the roll type with bottles or bottles with bottles, orbital stirrer for Erlenmees, with or without baffles (shaken flash), toggle stirrer (wave), a bioreactor with pneumatic stirring (air-lift) or a rotary blade stirrer such as a marine propeller type stirrer, Rushton turbine, stirring anchors, barrier stirrer, helical ribbons. A preferred rotary stirrer is a vertical blade turbine. Finally, static structures may be present in the container (4), for example baffles, or structures forming partial barriers to liquid movement, such as those used in a static mixer, may naturally also be used. The stirrer (7) and the dimensions of the container (4) are adapted to control a turbulent flow of the liquid medium (5) in the container (4). A person skilled in the art of his general knowledge knows how to calculate the length of Kolmogorov Lₖ adapted for each type of stirrer (7) as a function of the dimensions of the container (4), the geometry of the stirrer (7) and the intensity of the agitation. The term “turbulent flow” means a flow whose Reynolds number Re is greater than 2000. The Reynolds number may for example be calculated by formula (IV). Preferably, the Reynolds number Re of the liquid medium flow (5) is greater than 7 000, preferably at 10 000 and preferentially at 12 000.

Other stirrers (7) for controlling a turbulent flow according to the present invention are well-known stirrers of a person skilled in the art and capable of being implanted in the system according to the present invention.

The stirrer (7) used in the exemplary embodiments of the invention comprises a blade such as a blade wheel arranged in a container 4 and moved by a magnetic force transmission system. The speed of the blade in the liquid medium (5) causes a flow of the liquid medium (5). The stirrer is adapted to control a flow, which, in view of the dimensions of the container (4), is turbulent. In the case of the stirrer (7) illustrated in FIG. 1 or 2, several parameters make it possible to calculate a value representative of the turbulence of the liquid medium (5), in particular the kinematic viscosity v of the liquid medium (5), the dimensions of the container (4) and in particular the volume v of liquid medium (5) contained in the container (4), the number of power Nₚ corresponding to the submerged part of the blade, the diameter D of the stirrer and in particular of the wheel, the speed N of rotation of the wheel. The user can thus calculate, as a function of these parameters, values representative of the turbulence of the flow, and in particular the length of Kolmogorov Lₖ, as given by equations (I), (II) and (III). In particular, the stirrer (7) is adapted to control a flow in which the length Lₖ is less than 100 μm, preferably less than or equal to 80 μm. More preferably, the stirrer 7 is adapted to control a flow in which the length Lₖ is less than or equal to 70 μm and very preferentially less than or equal to 60 μm. In a particularly preferred manner, the flow has a Lₖ less than or equal to 55 μm, also preferably less than or equal to 50 μm.

In an exemplary embodiment of the fluidic system (1), the speed of rotation of the stirrer (7) is capable of being controlled at 100 rpm (rotations per minute), the diameter of a blade such as, for example, a blade wheel is 10.8 cm and the volume of liquid medium contained by the container (4) is 400 mL. The measured number of power N_{P} of the blade in the liquid medium 5, by formula (III), is substantially equal to 3.2. The energy dissipated per unit of mass E, calculated by formula (II), is equal to 5.44·10⁻¹·J·kg⁻¹. The length of Kolmogorov Lₖ calculated by formula (I) is thus equal to 41.8 μm.

### Preparation of Microcarriers and Producer Cells

The container (4) can be disposable or sterilized prior to any introduction of liquid medium (5), microcarriers (3), producer cells (6) and the therapeutic agent or imaging agent. The microcarriers (3), in the occurrence of the microbeads (14), are also sterilized. The microbeads (14) are incubated in the culture medium of the producer cells (6), comprising serum, in the container (4). This incubation makes it possible to oxygenate the culture medium and to cover the surface of the microbeads (14) of a layer, at least partially, of proteins, promoting the adhesion of the producer cells (6) to the surface of the microbeads (14).

The producer cells (6), before being introduced into the fluidic system (1), are suspended by means of a medium comprising trypsin. They can then be centrifuged at 300 g for five minutes to be concentrated in the base of a tube, so as to replace the medium comprising trypsin by a DMEM medium. The producer cells (6) are then introduced into the container (4), comprising culture medium and the microbeads (14), in an amount corresponding substantially to 5 to 20 producer cells (6) per microbead (14). The producer cells (6) and the microbeads (14) are then agitated and then sedimented, so as to contact the microbeads (14) and the producer cells (6), and promote the adhesion of the producer cells (6) to the surface of the microbeads (14). The agitation can resume periodically, so as to promote the homogeneity of the adhesion of the producer cells (6) to the surface of the microbeads (14), for example every 45 minutes for 5 to 24 hours. The culture of the producer cells is then carried out with a low agitation of the culture medium (for example the rotation of a blade such as a blade wheel at a speed of 20 rpm), as well as a regular replacement of the culture medium (for example a replacement of 5%, 10%, 15%, 20%, 25%, 30%, 35% or 40% of the culture medium each day).

### The Loading of a Therapeutic or Imaging Agent

The fluidic system (1) for the loading and production of extracellular vesicles (EV) is intended for the large quantity production of extracellular vesicles (EV) in a container (4). However, the invention is not limited to this embodiment and also allows for large quantities of the therapeutic agents and/or imaging agents in the extracellular vesicles (EV) produced according to the invention. Thus, the producer cells (6) and the therapeutic or imaging agent are simultaneously suspended in the liquid medium (5) and mixed in the container (4). Alternatively, the producer cells (6) can be added sequentially in the liquid medium (5), i.e., before or after the addition of the therapeutic agents and/or imaging agents in said liquid medium (5). In general, any type of therapeutic and/or imaging agent can be used, wherein the therapeutic agents may in particular be molecules or particles for treating infectious diseases, inflammatory diseases, metabolic diseases, degenerative diseases, traumatic diseases, post-surgical diseases, genetic diseases, malignant tumors, orphan diseases, diseases of the vasculature, diseases of the lymphatic system, diseases of the locomotor system, diseases of the digestive system, diseases of the nervous system, diseases of the reproductive system, diseases of the excretory system and/or agents (molecules or particles) of nuclear, magnetic, optical, acoustic. The container (4) also comprises a stirrer (7) as described above and for agitating the liquid medium (5) comprising the suspension-producing cells (6) and the therapeutic or imaging agent. Preferably, the fluidic system (1) is adapted to generate a sufficiently gentle agitation to homogenize the medium without damaging the producer cells but sufficient to induce shear stresses in the liquid medium (5) interior of the container (4) in order to effectively charge the therapeutic and/or imaging agents in the producer cells (6) and in the extracellular vesicles.

According to another object, the invention also relates to a method for ex vivo production of extracellular vesicles from producer cells (6), comprising:
controlling the speed of a stirrer (7) causing a turbulent flow of a liquid medium (5) in the container (4) to exert shear stresses on the producer cells (6) in order to carry out the loading and production of a therapeutic and/or imaging agent in the lumen of the extracellular vesicles (EV), the length of Kolmogorov of the flow being less than 100 μm in a container (4), the container comprising an outlet (9), the liquid medium 5 comprising producer cells (6), and
a collection of the liquid medium (5) comprising extracellular vesicles (EV), for example at the outlet (9) of the container (4). The collection may alternatively be carried out by transferring the whole of the liquid (5) contained in the container in another container. Optionally, a centrifugation the speed of which is adapted to separate the extracellular vesicles on the one hand and the producer cells and/or the adherent producer cells on the microcarriers on the other hand is applied.
   In a particular embodiment, the length of Kolmogorov of the flow is less than or equal to 80 μm, or even less than or equal to 70 μm and, preferably, less than or equal to 60 μm. In a particularly preferred mode, said Kolmogorov length is less than or equal to 55 μm. In a preferred embodiment, said Kolmogorov length is less than or equal to 50 μm.
   According to an object of the invention, the method according to the invention comprises a step of loading a therapeutic and/or imaging agent. Of course, this step can also be carried out before the step of producing extracellular vesicles. In this particular embodiment, the vesicle-producing cells are thus loaded in their membrane and/or their cytoplasm as a therapeutic and/or imaging agent of interest prior to the implementation of the method for producing extracellular vesicles. The loading is carried out either by known methods of the art, such as for example passive loading or, in a preferred manner, according to the method for loading at least one therapeutic and/or imaging agent into the membrane or in the cytoplasm of producer cells (6), comprising:
A control of the speed of an stirrer (7) causing a turbulent flow of a liquid medium (5) in a container (4) to exert shear stresses on the producer cells (6) in order to carry out the loading of a therapeutic and/or imaging agent into the membrane or in the cytoplasm of the producer cells (6), the length of Kolmogorov of the flow being less than or equal to 100 μm, in a container (4), the container comprising an outlet (9), the liquid medium (5) comprising the therapeutic and/or imaging agent, producing cells (6), and
Collection of the liquid medium (5) comprising extracellular vesicles (EV) at the outlet (9) of the container (4), and
optionally, a collection of the charged producer cells.

According to a particular embodiment, the length of Kolmogorov of the flow is less than or equal to 80 μm, or even less than or equal to 70 μm and, preferably, less than or equal to 60 μm. According to a still more particular characteristic, Lₖ is less than or equal to 55 μm. According to another particular characteristic, Lₖ is less than or equal to 50 μm.

Alternatively, in another embodiment, the loading step may be carried out after the step of producing extracellular vesicles. This embodiment may be of interest in the case where it is desired to obtain a 1^{st} production of uncharged vesicles followed by a 2^{nd} production of extracellular vesicles loaded with said therapeutic and/or imaging agent, and this in the context of placing a fluidic system with a collection of the liquid medium (5) continuously. In this embodiment, once produced, in a manner independent of their production, the vesicles are loaded by being subjected to shear stresses by controlling the speed of an stirrer (7) resulting in a turbulent flow of a liquid medium (5) in the container (4) in which are the extracellular vesicles in order to achieve the same in the light or membrane of the extracellular vesicles (EV), of a therapeutic and/or imaging agent also contained in the liquid medium (5), the length of Kolmogorov of the flow being less than 100 μm in a container (4). According to a particular feature, the length of Kolmogorov of the flow is less than or equal to 80 μm, or even less than or equal to 70 μm and, preferably, less than or equal to 60 μm. According to a particularly preferred feature, Lₖ is less than or equal to 55 μm. According to a further preferred feature, Lₖ is less than or equal to 50 μm.

Surprisingly, as demonstrated in the experimental part, the flow that allows the producer cells (6) to produce extracellular vesicles also allows and simultaneously to charge the therapeutic or imaging agent in the producer cells (6) and therefore produce said extracellular vesicles (EV) in a container (4) loaded with the therapeutic and/or imaging agent. Thus, alternatively, and in another embodiment, the step of loading said therapeutic and/or imaging agent is simultaneous to the step of producing extracellular vesicles. An object of the invention is therefore also the method for loading at least one therapeutic and/or imaging agent into the membrane and/or in the cytoplasm of producer cells (6) and/or in the membrane or the light of the extracellular vesicles of said cells, comprising:
A control of the speed of an stirrer (7) causes a turbulent flow of a liquid medium (5) in a container (4) to exert shear stresses on the producer cells (6) and the extracellular vesicles in order to carry out the loading of a therapeutic and/or imaging agent into the membrane or in the cytoplasm of the producer cells (6) and the light and/or membrane of the vesicles of said cells, the length of Kolmogorov of the flow being less than or equal to 100 μm, in a container (4), the container comprising an outlet (9), the liquid medium (5) comprising the therapeutic and/or imaging agent, producing cells (6), and
Collection of the liquid medium (5) comprising extracellular vesicles (EV) at the outlet (9) of the container (4).
   According to a particular embodiment, the length of Kolmogorov of the flow is less than or equal to 80 μm, or even less than or equal to 70 μm and, preferably, less than or equal to 60 μm. According to one even more particularly characteristic, Lₖ is less than or equal to 55 μm. According to another also particular characteristic, Lₖ is less than or equal to 50 μm.

Preferably the extracellular vesicles (EV) at the outlet (9) of the container (4) comprise a mixture of extracellular vesicles loaded with a therapeutic and/or imaging agent and extracellular vesicles not loaded with a therapeutic and/or imaging agent.

Example of the Production of EV Extracellular Vesicles with Loading of Therapeutic Agent or Imaging Agent

The extracellular vesicles (EV) are produced in a container (4) containing a liquid medium (5), for example without serum, of the producer cells (6). The medium used before the production for the culture of producer cells (6) comprising serum and therapeutic and/or imaging agents, three to four times the container (4) is washed with liquid medium 5 DMEM without serum, each washing corresponding for example to a volume of approximately 400 mL. The stirring of the liquid medium (5) is then controlled by the stirrer (7) so as to cause a turbulent flow in the container (4). The agitation is preferably adjusted so as to control a flow of the liquid medium (5) in which the length of Kolmogorov Lₖ is less than 100 μm and preferentially less than or equal to 60 μm. The agitation of the liquid medium (5) is controlled at least for 20 minutes, preferably for more than one hour, and preferably for more than two hours. According to a particular aspect, the stirring lasts two hours. The loading and production of charged extracellular vesicles (EV) can be measured during production. To this end, the agitation can be momentarily interrupted. The producer cells 6 are allowed to settle and/or centrifuged at the bottom of the container (4), then a liquid medium sample 5 comprising extracellular vesicles (EV) is taken. Centrifugation of the sample is carried out at 2000 g for 10 minutes, so as to remove cell debris. The supernatant is analysed by a method for individual tracking of particles (or NTA, acronym English of Nanoparticle Tracking Analysis) so as to count the number of extracellular vesicles (EV) and to deduce therefrom the concentration of extracellular vesicles (EV) of the samples. It can be verified that the concentration of extracellular vesicles (EV) at the beginning of the agitation is close to zero or negligible.

The extracellular vesicles (EV) produced can also be observed and/or counted by transmission electron microscopy. To this end, a drop of 2.7 μL of solution comprising EV extracellular vesicles is deposited on a grid suitable for cryo-microscopy, then immersed in liquid ethane, resulting in near-instantaneous freezing of said drop, avoiding the formation of ice crystals. The grid supporting the extracellular vesicles (EV) is introduced into the microscope and the extracellular vesicles (EV) are observed at a temperature of the order of −170° C.

### Extracellular Vesicle Separation

The extracellular vesicles (EV) loaded and produced in the container 4 are capable of being extracted from the container (4) by the outlet (9) of the container (4), suspended in the liquid medium (5). A filter (18) can be arranged at the outlet (9) so as to filter the producer cells 6 and the cell debris upon extraction of extracellular vesicles EV from the container (4). A connector (13) is fluidically connected to the outlet (9), allowing the transport of the liquid medium (5) comprising the produced extracellular vesicles (EV).

The fluidic system (1) may further comprise a separator (15) of extracellular vesicles (EV). The separator (15) comprises an inlet of the separator (10), in which the liquid medium (5) comprising extracellular vesicles (EV) from the container (4) can be fed directly or indirectly. The separator (15) may also comprise a first outlet (11) of the separator, through which the liquid medium (5) is able to exit the separator (15) with a concentration of EV extracellular vesicles smaller than at the inlet (10) of the separator (15), or even substantially zero. The separator (15) may also comprise a second outlet (12) of the separator (15), through which the liquid medium (5) is capable of exiting the separator (15) with a higher concentration of extracellular vesicles (EV) than at the inlet (10) of the separator (15).

In general, the separator (15) of EV extracellular vesicles can be fluidly connected to the container (4) so as to be capable of reintroducing a liquid medium (5) depleted in EV vesicles into the container (4), for example by the inlet (8) of the container (4). Thus, the production and/or extraction of charged extracellular vesicles (EV) can be carried out continuously, with a substantially constant volume of liquid medium (5) in the container (4). According to an alternative embodiment, the fluidic system does not comprise a separator (15) of extracellular vesicles (EV) or the fluidic system comprises a separator (15) of extracellular vesicles (EV) that can be fluidly connected or not, for example via a means for closing said separator (15), to the container (4). Thus, the production and/or extraction of charged extracellular vesicles (EV) can be carried out in a discontinuous or continuous manner according to the opening or closing of the closing means arranged upstream of the separator (15).

In the exemplary embodiment of a fluidic system (1) illustrated in FIG. 1 or 2, the liquid medium (5) can be extracted from the container 4 by a first pump (16), via a connector (13), so as to transport the liquid medium (5) into a collector (19). Another pump (16′) makes it possible to supply the liquid medium (5) contained in the collector (19) to the inlet (10) of the separator (15), via another connector. The first outlet (11) of the separator (15) is connected to the container 4 via a connector, so as to reintroduce liquid medium (5) depleted in extracellular vesicles (EV) into the container (4). The second outlet (12) of the separator (15) is connected to the collector (19) via a connector, so as to enrich the liquid medium (5) contained in the collector (19) into extracellular vesicles (EV). Alternatively, the inlet (10) of the separator (15) can be directly connected to the outlet (9) of the container (4) (or via a first pump (16)). The first outlet (11) of the separator (15) is connected to the container (4) and the second outlet (12) of the separator (15) is connected to the collector 19. Several separators may also be arranged in series to vary the degree of separation of extracellular vesicles (EV) in the liquid medium (5), and/or in parallel to adapt the flow rate of liquid medium 5 in each separator (15) to the flow rate of a first pump (16).

### Influence of the Agitation on the Loading of the Extracellular Vesicles EV

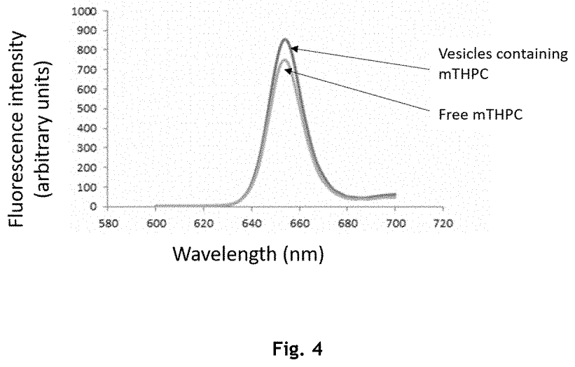

FIG. 3 illustrates the size distribution of EVs obtained by NTA (Nanoparticle Tracking Analysis, NS300, Malvern) (A) and the morphological Analysis by cryo-TEM (Cryo Transmission Electron Microscopy (B). The size distribution of the turbulence-triggered EVs from the HUVEC was analysed by NTA and cryo-TEM (FIG. 3) showing the form of the vesicles and the size range of the polydispersed EVs (C). The results show that EVs obtained at a Kolmogorov length of 35 μm have a conventional size range (100 to 400 nm). The average size of EVs were respectively 236 nm and 200 nm. The presence of mTHPC (meta-tetra (hydroxyphenyl) chlorin, INN: tempo, temoporfin in French) in the fraction of the isolated EVs is demonstrated by fluorometry with an emission peak to 650 nm characteristic of this molecule (following excitation to 400-410 nm) (FIG. 4).

The quantification of mTHPC was carried out for samples of EVs produced by producer cells incubated with 100 μM mTHPC under stirring. Loading experiments were carried out at a length of Kolmogorov of 100 and μm in order to determine the effect of turbulence on the internalization of the agents of interest on the producer cells and subsequently on the released EVs. In order to establish a comparison with an equivalent quantity of EVs, the loading step at 100 μm was followed by washing and a 35 μm-length vesicle of length Kolmogorov, according to the experimental protocol of Table 1 below.

TABLE 1
protocol for loading HUVEC vesicles to mTHPC

| | | |
|---|---|---|
| Duration | Loading at Lₖ = 100 μm, | Loading and vesiculation |
| | followed by a vesiculation | at Lₖ = 35 μm |
| | at Lₖ = 35 μm | |
| 2 h | Lₖ = 100 μm | Lₖ = 35 μm |
| | DMEM + mTHPC 100 μM | DMEM + mTHPC 100 μM |

Washing 4 times (DMEM without serum)

| | | |
|---|---|---|
| 2 h | Lₖ = 35 μm, DMEM | — |

The purified EVs samples (EV) obtained as a result of a load of the cells at a length of Kolmogorov of 100 and 35 μm contained a concentration mTHPC of 1.3 mM and 7.8 mM, respectively, which means an increase in more than 5 times of the charge of EVs with the mTHPC. Moreover, when the amount of EV obtained at 35 μm to 100 μm is compared, an increase of times, testing the effect and the importance of the length of Kolmogorov for, on the one hand, triggering and increasing the release of EVs and, on the other hand, also increasing their loading into a cargo molecule of interest.

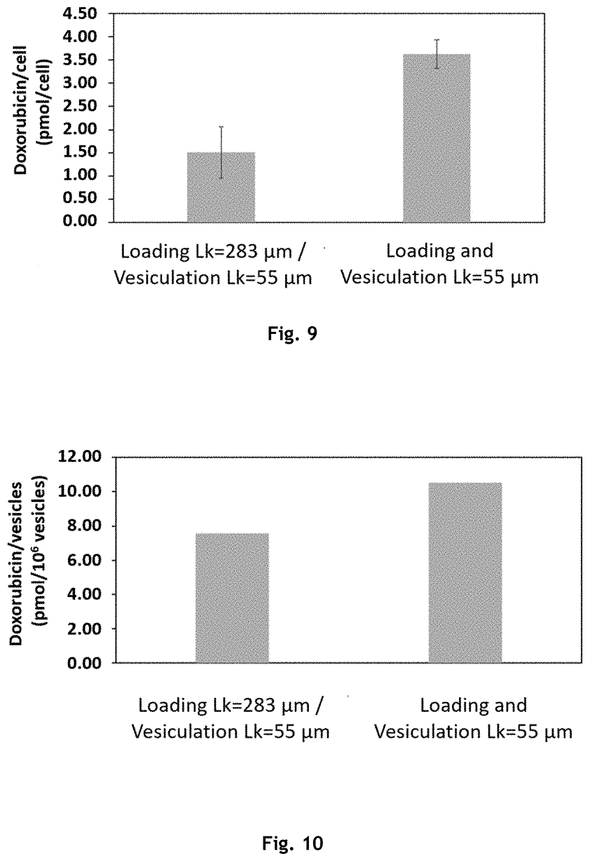

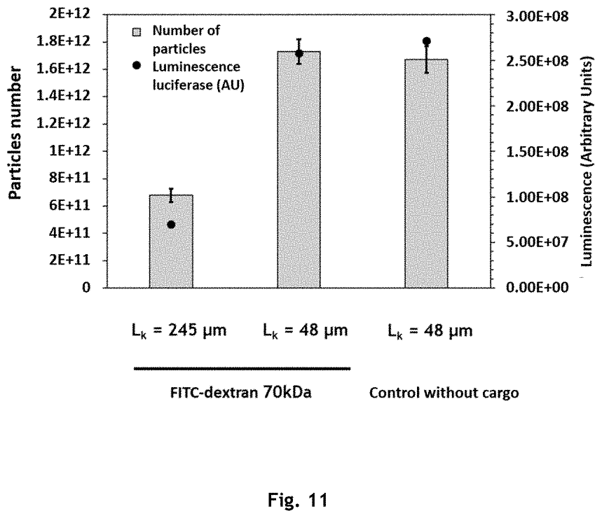

The same experiment was carried out using HUVEC (ATCC, FIGS. 9 and 10) and murine mesenchymal stem cells (CSM) (C3HT1/2, ATCC) using different Lₖ. Briefly, the cells were cultured in DMEM containing 10% fetal calf serum (SVF) and 1% penicillin-streptomycin (PenStrep, Gibbco) at 37° C. (5% CO₂). They have been seeded on Cydex 1 microcarriers (GE Healthcare) at 12 g of beads/L in micro-carrier spinner flashes (Bellco) of 100 mL, at 13300 cells/cm², then cultured at 6 g of beads/L with stirring of 34 rpm until confluence. Prior to the loading of doxorubin, the microcarriers comprising the confluent cells were washed 3 times with DMEM in order to remove any trace of serum. After balancing in serum-free DMEM, doxorubicin was added at a final concentration of 5 mM. The loading and vesiculating were carried out at 37° C. (5% CO₂) according to the protocol of Table 2 below.

TABLE 2
protocol for loading HUVEC or CSM murine vesicles to doxorubicin

| | | |
|---|---|---|
| Duration | Loading at Lₖ = 283 μm | Loading and vesiculation |
| | (passive loading) then | at Lₖ = 55 μm |
| | vesiculation | |
| 2 h | Lₖ = 283 μm | Lₖ = 55 μm |
| | DMEM + | DMEM + |
| | Doxorubicine 5 mM | Doxorubicine 5 mM |

Washing 4 times (DMEM without serum)

| | | |
|---|---|---|
| Vesiculation | Lₖ = 55 μm, DMEM | — |
| 2 h | | |

At the end of culture, the cells were isolated from the beads by trypsin 10 min at 37° C. (5% CO₂), and separated from the beads by passage on cell sieve 70 μm, then washed in PBS to remove free doxorubicin. After a first step of centrifugation at 2000 g for 10 minutes, the vesicles were isolated and concentrated by ultracentrifugation (Beckman Optics MAX XP, 150 000 g for 1 h30). The cell concentration was determined with the NC200 cell counter (Chemometec) and the concentration and size distribution of the vesicles were calculated by NTA. The markers present in the vesicles or their membrane were analysed by flow cytometry using the MACSPlex kit (Miltenyi Biotec). Vesicles and cells were then chemically lysed (Triton 0.3%) and then analyzed to the fluorescence spectrophotometer (Hitachi F7000). Doxorubicin was quantified using its excitation wavelengths at 485 nm and emission at 560 nm.

FIG. 9 confirms the importance of Lₖ in the loading of HUVEC on the one hand: the loading of the HUVEC cells is 2.4 times greater (+140%) using a Lk of 55 μm than at a Lk of 283 μm, and on the other hand in the loading of the vesicles (FIG. 10): an increase in 39% of the doxorubicin concentration of the vesicles is observed when a Lk of 55 μm is used, with respect to a Lₖ of 283 μm, which corresponds to a passive loading of the cells or vesicles (FIG. 10).

Similar results are obtained for murine MSCs (not shown) with in particular an increase in 485% of the concentration of doxorubicin vesicles when a Lₖ of 55 μm is used, with respect to a passive loading at a Lₖ of 283 μm. The loading of the cells is also increased by 592%.

Regarding the characteristics of the vesicles produced, the NTA data shows a similar size distribution between the loading conditions (passive and for a Lk=55 μm), respectively 106.9 nm and 109.7 nm for HUVEC vesicles, which correspond to a conventional size of extracellular vesicles (not shown). The flow cytometry analysis shows that the vesicles obtained according to the two conditions have conventional markers of extracellular vesicles including CD9, CD63 and CD81 (not shown). Thus, the vesiculating at a Lₖ of less than 40 μm has no impact on the presence of conventional vesicles of the vesicles; it is therefore possible to wait for a vectorization functionality at least as high as the vesicles produced passively.

The results show the influence of Lₖ in the production and loading of vesicles on different types of cells used. mTHPC and Doxorubicin are small therapeutic agents (respectively 680.7 and 543.5 kDa). The conditions identified by the inventors are more effective than the passive loading condition without agitation or at very low stirring at 283 μm, for example. The effect of the variation of Lₖ for the production of extracellular vesicles for larger agents was also tested (FIGS. 11, 12, 13). Dextran-FITC probes of kDa and 70 kDa were used. They may be considered as model of imaging agents and also as models for therapeutic agents that would be of greater molecular weight, such as, for example, polymers, siRNAs which have a molecular weight of the order of 13 kDa (Whitehead et al, 2009) or “small” therapeutic proteins whose molecular weight is close to 70 kDa (Strohl, 2015). Similar results are obtained only for dextran-FITC probes of 70 kDa or 10 kDa.

Briefly, HeLa (ATCC) cells, genetically engineered to express HSP70 protein-bound luciferase (HSP70 is a marker of extracellular vesicles, Thery and al 2018), were cultured with DMEM containing 10% fetal calf serum (SVF) and 1% penicillin-streptomycin (PenStrep, Gibbco) at 37° C. (5% CO₂). These cells were seeded on Cytodex 1 microcarriers (GE Healthcare) to 6 g of beads/L by Spinner Flash (Bellco) of 100 mL at 6700 cells/cm², then cultured at 3 g of beads/L with stirring of 34 rpm until confluence. Prior to loading, the microcarriers comprising the confluent cells were washed 3 times with serum-free DMEM to remove traces of serum and then incubated with FITC-dextran probes of 10 or 70 kDa (FD10S references and 90718 respectively, Sigma) at 1.43 mM for 2 hours at Lₖ of 245 or 48 μm (table 3).

TABLE 3
protocol for production and loading of vesicles of HeLa cells
with dextran 10 or 70 kDa coupled to the FITC.

| | Passive loading and | Loading and vesiculation |
|---|---|---|
| | vesiculation | according to the invention |
| Duration | Lₖ = 245 μm | Lₖ = 48 μm |
| 2 h | DMEM + | DMEM + |
| | FITC-dextran | FITC-dextran |
| | (10 or 70 kDa) | (10 ou 70 kDa) |
| | 1.43 mM | 1.43 mM |

After a first centrifugation to remove cellular debris (2 000 g for 10 minutes), and ultracentrifugation (Beckman Optics MAX XP, 110 000 g for 1 h). Cell concentration was determined with NC200 cell counter (Chemometec), and the concentration and size distribution of the vesicles was calculated by NTA (NS300, Malvern). The luminescent signal of the vesicles was analyzed by a plate reader (EnSworst, PerkinElmer). Vesicles and cells were then chemically lysed (Triton 0.3%) and the amount of FITC determined to the fluorescence spectrophotometer (Hitachi F7000), using the excitation wavelengths at 495 nm, and emission at 520 nm of the FITC. The extracellular vesicles obtained were also analyzed in flow cytometry imaging (Amnis® ImageStream®) using anti-CD 63 PE and anti-CD 81 APC (Biolegend) antibodies. In total, 100 000 events were analyzed. The positive events to the FITC labeling were then classified into the corresponding apoptotic bodies (AB), large vesicles (lEVs) and small vesicles (sEV) as a function of their relative granularity or internal complexity (“side scatter”).

The luminescent (luciferase) and fluorescent signal (FITC) for the vesicles obtained at a Lₖ of 245 μm and 48 μm using the 70 kDa FITC-dextran probe was evaluated. The luminescent signal is indicative of the number of vesicles produced by the HeLa cells, it corresponds to the luciferase linked to the HSP70 protein, which is a cytosolic marker of the vesicles, and produced by the mother cells. The fluorescence of the FITC reflects the amount of ITC-dextran internalized into the vesicles and thus the charge of the FITC-dextran vesicles.

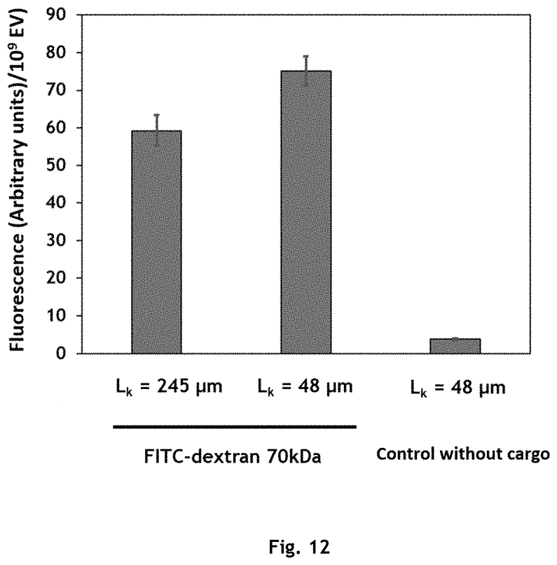

Regarding the production of vesicles with the 70 kDa FITC-dextran (FIG. 11), an increase in the production of vesicles of a factor 2 using a Lₖ of 48 μm with respect to the cells loaded at a Lₖ of 245 μm is observed, whether in NTA or a luminescent signal (which is found proportional to the number of vesicles counted in NTA). A control condition was prepared at a L_{K} of 48 μm but in the absence of the 70 kDa FITC-dextran probe. The same increase in the number of vesicles is observed in the absence of the 70 kDa cargo. Furthermore, the analysis of the fluorescent signal, indicative of the presence of the 70 kDa FITC-dextran probe, indicates, with equal number of vesicles, an increase in 27% of the fluorescent signal (FIG. 12), thus signalling a larger loading of the vesicles when Lₖ=48 μm, with respect to the conditions where Lₖ=245 μm.

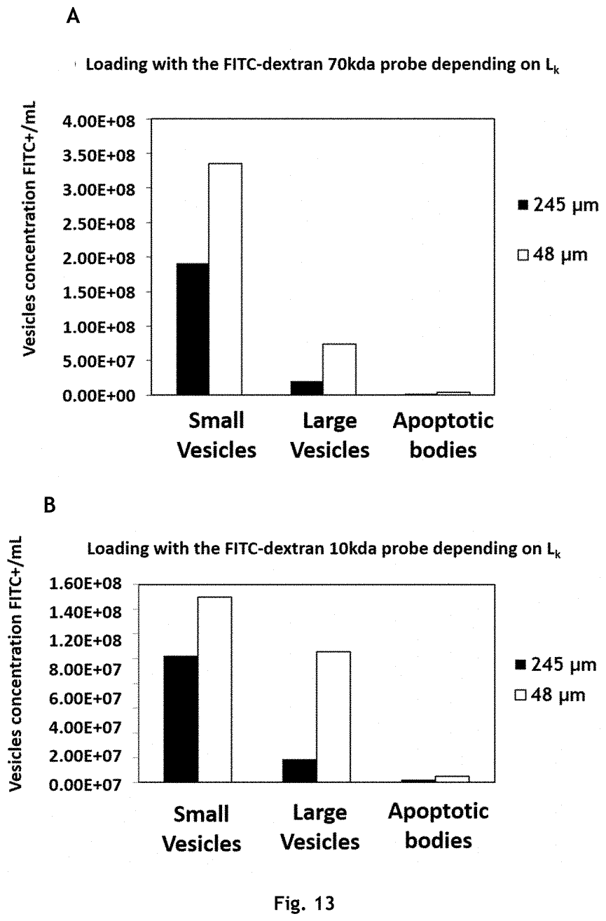

The increase in loading efficiency is observed regardless of the size of the extracellular vesicle produced by the cells (small vesicles, large vesicle, apoptotic body). The different types of vesicles and their marking were visualized by imaging in flow cytometry (ImageStream®, not shown) indicating the presence of the fluorescent markers CD81 and CD63 and making it possible to confirm that the NTA data well correspond to extracellular vesicles and not aggregates of cell debris. The flow cytometry analysis (by plotting the intensity of the side scatt and the marking intensity FITC) is indicative of their loading by the probes FITC-dextran (FIGS. 13 A and B). The encrypted results of increasing the loading of the extracellular vesicles when a Lₖ of less than 100 mM is used are carried out in Table 4 below:

TABLE 4

| | Increased cargo loading | |
|---|---|---|
| | (Lk = 48 μm vs Lk = 245 μm) | |
| Vesicle size | FITC-dextran 10 kDa | FITC-dextran 70 kDa |
| Small EVs | +75.0% | +46.3% |
| Large EVs | +263.1% | +460.0% |
| Apoptotic bodies | +52.6% | +92.0 |

Generally, flow cytometry analysis for labeling with anti-CD 63 PE and anti-CD 81 APC antibodies shows a substantial number of extracellular vesicles labelled with either antibody (not shown). Thus, the increase in the production of extracellular vesicles does not occur at the detriment of the vesicle quality in terms of the presence of conventional extracellular vesicles markers.

These results show that the choice of a length of Kolmogorov (Lₖ) according to the invention makes it possible to increase the yield of cell vesicle production HeLa, that the cargo has no influence on this yield and also a significant effect of Lₖ in the loading of the vesicles. It therefore makes it possible to produce, in addition to the type of cells (primary cultures or immortalized cells, stem cells, endothelial, epithelial, human or animal cells) and over a wide range of cargo size.

It is derived from the set of results that the choice of a length of suitable Kolmogorov, less than 100 μm, makes it possible to significantly optimize the production and loading of extracellular vesicles regardless of the size of the intended cargo and the type of cells. Thus, the extracellular vesicles produced according to the invention have a higher concentration of imaging agent and/or a therapeutic agent than the extracellular vesicles produced according to the currently known methods, such as passive loading (without or at very low stirring). Furthermore, the method according to the invention does not comprise the application of an electrical stress on the cells via the application of a potential difference such as the electroporation method.

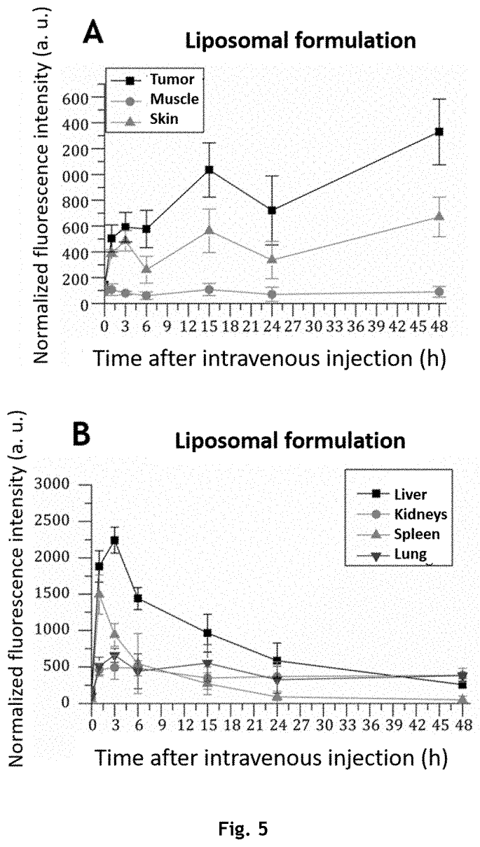

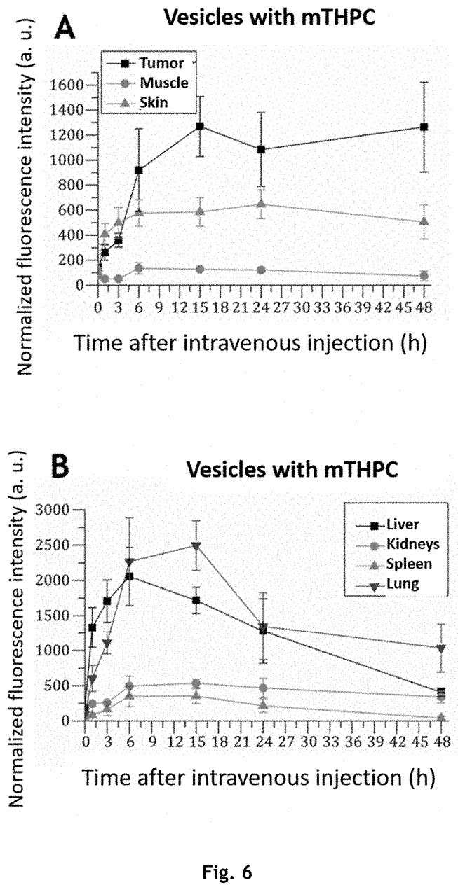

FIGS. 5 and 6 illustrate the biodistribution of a liposomal formulation of mTHPC (FIG. 5) and mTHPC-EV (FIG. 6) as a function of the intensity of fluorescence in selected tissues as a function of time after intravenous injection (0.3 mg/kg of the agent of interest) in mice carrying HT29 tumors. Becoming EVs mTHPC at a length of Kolmogorov of 35 μm was studied in a mouse tumor model. The biodistribution following intravenous administration was compared to a liposomal formulation of mTHPC (a liposomal formulation mTHPC) by taking advantage of the imaging properties of the drug. The biodistribution data (FIGS. 5 and 6) indicate that the EVs mTHPC have reached a maximum concentration in the tumor faster (between 6 and 15 h after injection) than the liposomal formulation of mTHPC (between 24 and 48 h after injection). Pulmonary absorption was higher for the EVs mTHPC than for the liposomal formulation of mTHPC. Absorption in the liver as high for EVs mTHPC and the liposomal formulation of mTHPC was observed.

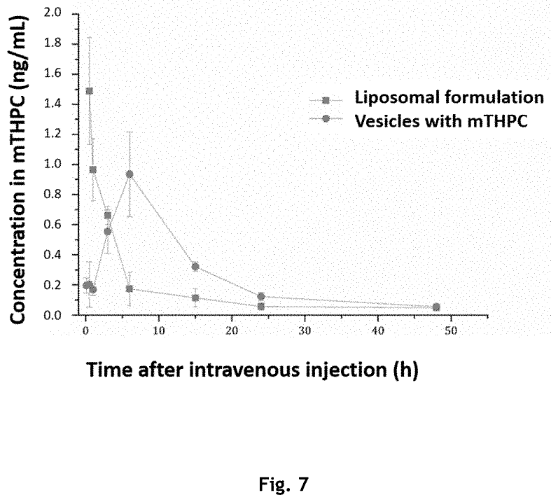

FIG. 7 illustrates the plasma concentration of mTHPC expressed as a function of time after intravenous injection of the liposomal formulation of mTHPC or mTHPC-EV (0.3 mg/kg mTHPC) in mice carrying HT29 tumors. A pharmacokinetic study revealed a decrease in mTHPC in the circulation after the injection of the liposomal formulation of mTHPC after a peak of 30 minutes after injection (FIG. 7). Conversely, the blood concentrations of mTHPC were surprisingly increased with a peak at 6 h after the injection. The decrease in the plasma concentrations of mTHPC near 0.2 ng/ml has reached 6 h and 24 h after the injection of the liposomal formulation of mTHPC and mTHPC, respectively.

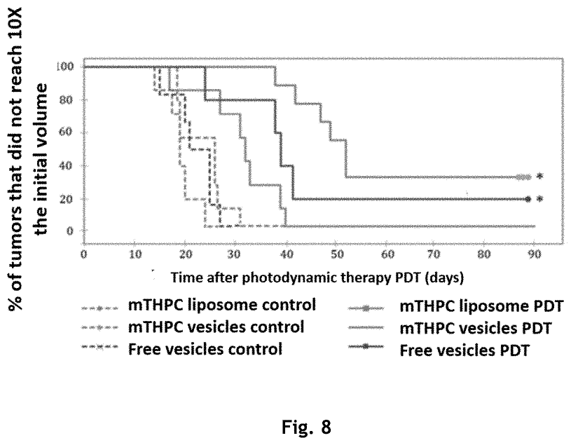

FIG. 8 illustrates Kaplan-Meier diagrams of tumor growth retardation HT29 after treatment with free mTHPC; the liposomal formulation of mTHPC and mTHPC-EVs with laser activation of the drug (photodynamic therapy, three-dimensional therapies), compared to the same groups without laser activation (control, dotted lines). The inventors have compared the therapeutic effect of EVs mTHPC, the liposomal formulation of mTHPC and free mTHPC in terms of tumor growth, 90 days after treatment. Kaplan-Meier diagrams show that, without laser-induced drug activation, mTHPC EV, the liposomal formulation of mTHPC and free mTHPC had the same therapeutic effect. However, photodynamic therapy for EVs mTHPC followed by laser activation results in an improved therapeutic effect with respect to the liposomal formulation of laser-activated mTHPC and free mTHPC. The results show that 0% of the control tumors or the group treated by the photodynamic therapy with the liposomal formulation of mTHPC, as well as 20% of the tumors treated by the photodynamic therapy to mTHPC, posters a growth 10 times higher, while this value was 33% for the group treated by the TDP of EV mTHPC at day 90 (FIG. 8). Thus, compared to the liposomes, the charged vesicles of active agents obtained according to the methods of the present invention can constitute a very advantageous alternative both of the point of view of the pharmacodynamics properties and the effectiveness of the treatment carried out. The liposomes are known for the vectorization of various biomolecules such as enzymes, hormones, antisense oligonucleotides, ribozymes, proteins or DNA peptides, cancer molecules (Farjadian et al 2018). Such molecules can therefore advantageously be vectorized by the vesicles loaded according to the method of the invention in the absence of mTHPC. Indeed, active agents in lipid formulations make the object of authorization to put on the market by the health authorities, for example:
amphotericin B (AmBisome®, allowed in the treatment of fungal infections),
daunorubicin (Daunoxome®, allowed in the treatment of Kaposi's sarcoma extensive or visceral),
rinotecan (Oniyde® allowed in the treatment of adenocarcinomas),
Vincristine (Marqibo® allowed in the treatment of Phi-negative acute lymphoblastic leukemia or lymphoblastic lymphoma),
cytarabine (DepoCyte®, allowed in the treatment of lymphomatous meningitis).
   A particular object of the invention is therefore a method for producing extracellular vesicles loaded by one of these molecules and as described in the present application in any one of its embodiments. The vesicles or cells advantageously loaded by these agents by said method also constitute objects of the present invention. A particular object is a loading method according to the invention, an extracellular vesicle or a cell loaded according to this method, characterized in that the therapeutic agent used in this method and loaded in the vesicles and/or producer cells, is selected from temoporfin, amphotericin B, daunorubicin, irinotecan, vincristine and cytarabine.

### REFERENCES

Alvarez-Erviti L, Seow Y, Yin H, Betts C, Lakhal S, Wood M J. Delivery of siRNA to the mouse brain by systemic injection of targeted exosomes. J Neuroinflammation. 2011 Nov. 28; 8:166.
Farjadian F, Ghasemi A, Gohari O, Roointan A, Karimi M, Hamblin M R. Nanopharmaceuticals and nanomedicines currently on the market: challenges and opportunities. Nanomedicine (Lond). 2019 January; 14(1):93-126.
Haney M J, Klyachko N L, Zhao Y, Gupta R, Plotnikova E G, He Z, Patel T, Piroyan A, Sokolsky M, Kabanov A V, Batrakova E V. Exosomes as drug delivery vehicles for Parkinson's disease therapy. J Control Release. 2015 Jun. 10; 207:18-30.
Kolmogorov, A. N., 1941, January, The local structure of turbulence in incompressible viscous fluid for very large Reynolds numbers, In Dokl. Akad. Nauk, SSSR, Vol. 30, No. 4, pp. 301-305.
Lamichhane T N, Raiker R S, Jay S M. Exogenous DNA Loading into Extracellular Vesicles via Electroporation is Size-Dependent and Enables Limited Gene Delivery. Mol Pharm. 2015 Oct. 5; 12(10):3650-7.
Nienow, A. W., Et Miles, D., 1971, Impeller power numbers in closed vessels, Industrial Et Engineering Chemistry Process Design and Development, 10(1), 41-43.
Shtam T A1, Kovalev R A, Varfolomeeva E Y, Makarov E M, Kil Y V, Filatov M V. Exosomes are natural carriers of exogenous siRNA to human cells in vitro. Cell Commun Signal. 2013 Nov. 18; 11:88.
Smyth T1, Petrova K, Payton N M, Persaud I, Redzic J S, Graner M W, Smith-Jones P, Anchordoquy T J. Surface functionalization of exosomes using click chemistry. Bioconjug Chem. 2014 Oct. 15; 25(10):1777-84.
Strohl W R. Fusion Proteins for Half-Life Extension of Biologics as a Strategy to Make Biobetters. BioDrugs. 2015; 29(4):215-239.
Tian Y, Li S, Song J, Ji T, Zhu M, Anderson G J, Wei J, Nie G. A doxorubicin delivery platform using engineered natural membrane vesicle exosomes for targeted tumor therapy. Biomaterials. 2014 February; 35(7):2383-90.
Théry C, Witwer K W, Aikawa E, et al. Minimal information for studies of extracellular vesicles 2018 (MISEV2018): a position statement of the International Society for Extracellular Vesicles and update of the MISEV2014 guidelines. J Extracell Vesicles. 2018; 7(1):1535750.
Whitehead, K. A., Langer, R., Et Anderson, D. G. (2009). Knocking down barriers: advances in siRNA delivery. Nature reviews Drug discovery, 8(2), 129.
Zhou, G., Kresta, S. M., 1996, Impact of tank geometry on the maximum turbulence energy dissipation rate for impellers, AIChE journal, 42(9), 2476-2490

1-19. (canceled)

20. A fluid system for loading at least one therapeutic and/or imaging agent into the membrane or in the lumen of extracellular vesicles from producer cells, comprising at least one container, a liquid medium contained by the container, producer cells, an stirrer of liquid medium suitable for the growth of the producer cells, wherein it also comprises means for controlling the speed of the stirrer and the stirrer and the dimensions of the container are adapted to generate a turbulent flow of the liquid medium in the container in order to exert shear stresses on the producer cells in order to carry out the loading of a therapeutic or imaging agent into the membrane or the lumen of the produced extracellular vesicles simultaneously by the fluidic system, the length of Kolmogorov of the flow being less than 100 μm.

21. The fluidic system according to claim 20, wherein the Kolmogorov length of the flow is less than or equal to 80 μm, preferably is less than or equal to 70 μm, even more preferably is less than or equal to 60 μm.

22. The fluid system according to claim 20, comprising an outlet and a connector connected to the outlet, the connector being capable of comprising liquid medium and extracellular vesicles.

23. The fluid system according to claim 20, wherein a stirrer of liquid medium is a rotary stirrer of which the rotation speed, shape and size are adapted, with the shape and dimensions of the container, to generate a turbulent flow of the liquid medium in the container.

24. The fluid system according to claim 20, comprising a separator of extracellular vesicles, fluidly connected to the container so as to be capable of reintroducing into the container a liquid medium depleted in extracellular vesicles.

25. A method for loading at least one therapeutic and/or imaging agent into the membrane or in the lumen of extracellular vesicles from producer cells, comprising:
controlling the speed of a stirrer causing a turbulent flow of a liquid medium in a container to exert shear stresses on the producer cells in order to carry out the loading of a therapeutic and/or imaging agent into the membrane or the lumen of the extracellular vesicles, the length of Kolmogorov of the flow being less than 100 μm, the container comprising an outlet, the liquid medium comprising the therapeutic and/or imaging agent, producer cells, and
collecting the liquid medium comprising extracellular vesicles at the outlet of the container.

26. A method for loading at least one therapeutic and/or imaging agent into the membrane or in the lumen of extracellular vesicles, comprising:
providing extracellular vesicles in the liquid medium,
controlling the speed of a stirrer causing a turbulent flow of a liquid medium in a container to exert shear stresses on the vesicles in order to carry out the loading of a therapeutic and/or imaging agent into the membrane or the lumen of the extracellular vesicles, the length of Kolmogorov of the flow being less than 100 μm, the liquid medium comprising the therapeutic and/or imaging agent.

27. A method of loading at least one therapeutic and/or imaging agent into the membrane or cytoplasm of producer cells, comprising:
controlling the speed of an stirrer causing a turbulent flow of a liquid medium in a container to exert shear stresses on the producer cells in order to carry out the loading of a therapeutic and/or imaging agent into the membrane or in the cytoplasm of the producer cells, the length of Kolmogorov of the flow being less than 100 μm, in a container, the container comprising an outlet, the liquid medium comprising the therapeutic and/or imaging agent, producer cells, and
collecting the liquid medium comprising extracellular vesicles at the outlet of the container, and
optionally, a collection of the charged producer cells.

28. The method according to claim 25, wherein the Kolmogorov length of the flow is less than or equal to 80 μm, preferably is less than or equal to 70 μm, even more preferably is less than or equal to 60 μm.

29. The method according to claim 25, wherein the liquid medium is stirred for more than twenty minutes. The method of any one of claims 6 to 10 wherein the stirrer is controlled to cause a flow of the liquid medium at constant, intermittent, increasing or decreasing intensity.

30. The method according to claim 25, wherein a separator depletes a portion of the liquid medium collected at the outlet of the container into extracellular vesicles, and wherein the portion of the liquid medium is reintroduced into the container.

31. Extracellular vesicle loaded with at least one therapeutic and/or imaging agent obtained by the implementation of the fluidic system according to claim 20, and/or by a method of loading at least one therapeutic and/or imaging agent into the membrane or in the lumen of extracellular vesicles from producer cells comprising:
controlling the speed of a stirrer causing a turbulent flow of a liquid medium in a container to exert shear stresses on the producer cells in order to carry out the loading of a therapeutic and/or imaging agent into the membrane or the lumen of the extracellular vesicles, the length of Kolmogorov of the flow being less than 100 μm, the container comprising an outlet, the liquid medium comprising the therapeutic and/or imaging agent, producer cells, and
collecting the liquid medium comprising extracellular vesicles at the outlet of the container.

32. Producer cell loaded with at least one therapeutic and/or imaging agent obtained by the implementation of the fluidic system according to claim 20, and/or by the method for loading at least one therapeutic and/or imaging agent into the membrane or in the cytoplasm of producer cells, comprising:
controlling the speed of an stirrer causing a turbulent flow of a liquid medium in a container to exert shear stresses on the producer cells in order to carry out the loading of a therapeutic and/or imaging agent into the membrane or in the cytoplasm of the producer cells, the length of Kolmogorov of the flow being less than 100 μm, in a container, the container comprising an outlet, the liquid medium comprising the therapeutic and/or imaging agent, producer cells, and
collecting the liquid medium comprising extracellular vesicles at the outlet of the container, and
optionally, a collection of the charged producer cells.

33. The extracellular vesicle loaded with at least one therapeutic and/or imaging agent according to claim 31 for use as a vector for the administration of said at least one imaging agent and/or at least one therapeutic agent.

34. The extracellular vesicle loaded with at least one therapeutic agent for use according to claim 33 in the therapy of infectious, inflammatory, immunological, metabolic, cancer, genetic, degenerative or secondary diseases in surgeries or trauma.

35. The method according to claim 25, wherein said therapeutic agent is selected from temoporfin, amphotericin B, daunorubicin, irinotecan, vincristine, cytarabine.

36. The extracellular vesicle according to claim 31, wherein said therapeutic agent is selected from temoporfin, amphotericin B, daunorubicin, irinotecan, vincristine, cytarabine.

37. The loaded extracellular vesicle of at least one imaging agent according to claim 33, as a vector for the administration of said at least one medical imaging agent, such as fluorescence imaging, luminescence, or imaging via detection of radioactive isotopes, contrast agents with magnetic, plasmonic, acoustic or radio-opaque properties.
